(19) United States
(12) Patent Application Publication
(10) Pub. No.: US 2010/0130955 A1
(43) Pub. Date: May 27, 2010

(54) REUSABLE ABSORBENT ARTICLE FOR AMBULATORY USERS

(76) Inventor: Renee J. Tice, Hillsboro, OR (US)

Correspondence Address: LACKENBACH SIEGEL, LLP, LACKENBACH SIEGEL BUILDING, 1 CHASE ROAD, SCARSDALE, NY, 10583, US

(21) Appl. No.: 12/625,028

(22) Filed: Nov. 24, 2009

Related U.S. Application Data
(60) Provisional application No. 61/117,447, filed on Nov. 24, 2008.

(51) Int. Cl. A61F 13/49

(52) U.S. Cl. 604/385.16

(57) ABSTRACT

The invention is a reusable, absorbent article, such as a diaper. The article comprises a main article assemblage and extensible hip panels attached to opposite sides of the posterior end of the assemblage. The assemblage further comprises: an impermeable exterior layer; a moisture transmitting, skin facing, layer; and, one or more layers of absorbent material secured between the exterior layer and the skin facing layer. The impermeable exterior layer is joined to the skin facing layer along the entire perimeter of the layer. A portion of the perimeter is joined with a groin contouring elastic member along each lateral edge of the central portion of the assemblage. The hip panels each further comprise a strip of woven material and a cut panel edge, together with a plurality of substantially parallel and evenly spaced strands of elastic thread, which are secured in place by corresponding stitching, and an edge stabilizing elastic member. The hip panels additionally comprise a corresponding snap facing attached to each hip panel. One or more snap fastener prongs are affixed to each of the snap facings; and, one or more snap fastener sockets corresponding to each snap fastener prong, are located on the assemblage along its lateral edges.

### CROSS REFERENCE TO RELATED APPLICATIONS

This application is related to, and claims priority from, U.S. Provisional Patent Application Ser. No. 61/117,447, filed Nov. 24, 2008, the entire contents of which is incorporated herein by reference.

### BACKGROUND OF THE INVENTION

1. Field of the Invention

The present invention relates to a reusable absorbent diaper or similar article having improved ease of use. More specifically, the present invention relates to a reusable diaper or similar article having its absorbent zone separated from two lateral resilient extensible zones. The extensible zones allowing ambulatory users to easily don and doff the diaper without creating unnecessary bulk under an outer clothing layer.

2. Description of the Related Art

The related art involves construction of a diaper or article in a number of ways. For instance, U.S. Pat. No. 6,605,071 for a Toddler's Disposable/Reusable Garment With Universal Disposable Liner/Diaper, issued Aug. 12, 2003 to Gray et al. (hereinafter “Gray”) teaches the use of a liner.

The waistband structure of the Gray diaper does teach a front-segment of a releasable front panel that can be construed as improving the ability to access and remove the liner of the diaper. However, The general construction of the Gray diaper does not anticipate the design of the present invention where the snappable front panel can be lowered to easily remove the diaper from the body of the wearer.

U.S. Pat. No. 5,897,545 for an Elastomeric Side Panel For Use With Convertible Absorbent Articles, issued Apr. 27, 1999 to Kline et al. (hereinafter “Kline”) utilizes a fastening system that is different from the snaps of the present invention.

Kline is not limited to a disposable diaper; but, its fastening system is distinguishable from the present invention in that Kline is utilizing a device resembling VELCRO® to make the diaper re-fastenable. The present invention uses snaps that are easier because of VELCRO's limitations in the wash cycle and in attracting unwanted adherents.

U.S. Pat. No. 6,254,583 for a Reusable And Protective Underwear Having Improved Containment Properties And Improved Fit And Construction Methods Thereof, issued Jul. 3, 2001 to Coates. (hereinafter referred to as “Coates”).

Coates teaches a re-usable underwear garment having a fluid containment sling that can hold a fluid absorbent pad. The use of the sling and pad is in contrast to the present invention which utilizes leg cuffs to effect containment.

U.S. Pat. No. 6,648,867 for a Disposable Diaper With Breathable And Liquid Resistant Side Flaps, issued Nov. 18, 2003 to Minato et al. (hereinafter “Minato”).

Minato teaches a disposable diaper as opposed to the reusable diaper of the present invention; and, the constructive elements of Minato do not appear to read on the present invention. Minato teaches several elements that are identical to other patents in the field (base elements: liquid pervious topsheet; liquid impervious backsheet; and, a liquid absorbent core), but finds its novelty in the side flaps that enhance breathability while maintaining liquid resistance.

What is not appreciated by the prior art is the ease of use in doffing and donning the diaper or article that is achieved by separating the absorbent zone from two lateral resilient extensible zones. These extensible zones allow ambulatory users to don and doff the article easily over their feet without creating unnecessary bulk that fits poorly under typical outer clothing layers. The extensible hip panels allow for a comfortable fit.

Accordingly, there is a need for an improved diaper or article that allows ease of use in doffing and donning the diaper or article that is achieved by separating the absorbent zone from two lateral resilient extensible zones.

### ASPECTS AND SUMMARY OF THE INVENTION

An object of the present invention is to provide an improved diaper or article that allows ease of use in doffing and donning the diaper or article that is achieved by separating the absorbent zone from two lateral resilient extensible zones.

Another object of the present invention is to provide extensible zones to allow ambulatory users to don and doff the article easily over their feet without creating unnecessary bulk that fits poorly under typical outer clothing layers.

According to an embodiment of the present invention there is provided a reusable, absorbent article such as a diaper for use by an ambulatory user. The article comprises: a main article assemblage having a posterior end and an anterior end; a first extensible hip panel attached to the right side of the posterior end; and, a second extensible hip panel attached to the left side of the posterior end. The main article assemblage further comprises: an impermeable exterior layer; a moisture transmitting, skin facing layer; and, one or more layers of absorbent material secured between the impermeable exterior layer and the moisture transmitting skin facing layer.

The impermeable exterior layer is joined to the moisture transmitting skin facing layer along the entire perimeter of the moisture transmitting skin facing layer. A portion of the entire perimeter is joined with a groin contouring elastic member along a first lateral edge and a second groin contouring elastic member along a second lateral edge of the central portion of the main article assemblage.

The hip panels each further comprise a strip of woven material, and a cut panel edge which can be folded under for a more secure seal. Additionally, the panels comprise a plurality of substantially parallel and evenly spaced strands of elastic thread, which are secured in place by a plurality of substantially parallel and evenly spaced stitching. Each of the stitches corresponds to each of the threads. There is also included an edge stabilizing elastic member. The stitching runs perpendicular to the main article assemblage. The elastic thread on the interior face of each of the hip panels is secured at the posterior end in a seam with the lateral posterior of the main article assemblage, and, at the anterior end in a seam with a corresponding snap facing.

The hip panels each further comprise a corresponding snap facing attached to the edge opposed to the main article assemblage of each hip panel. One or more snap fastener prongs are affixed to each of the snap facings; and, one or more snap fastener sockets corresponding to each snap fastener prong, is located on the main diaper assemblage along the anterior portion of its lateral edges. Each of the snap fastener prongs is affixed to a snap facing through only the outward facing layer.

In another embodiment of the present invention, there is disclosed, and claimed, a reusable, absorbent article for use by an ambulatory user. The article has a main diaper assemblage having an outermost layer and a skin-facing layer; and, further comprises a posterior end, and an anterior end opposed to the posterior end. There is a first extensible hip panel attached to the right side of the posterior end; and, a second extensible hip panel attached to the left side of the posterior end. Each of the first extensible hip panel and the second extensible hip panel are attached to the main diaper assemblage by corresponding attachment means.

Each of the corresponding attachment means further comprises a crimped edge of the first or said second extensible hip panel passing between the outermost layer and the skin-facing layer of the main diaper assemblage. A reinforcing seam tape is stitched to the crimped edge to prevent slippage or disassembly of the sealed edge.

The above, and other objects, features, and advantages of the present invention will become apparent from the following description read in conjunction with the accompanying drawings, in which like reference numerals designate the same elements.

### BRIEF DESCRIPTION OF THE DRAWINGS

FIG. 1 is a perspective view of the absorbent article prepared for step-in donning.

FIG. 2 is a top perspective view of the skin-facing side of the open absorbent article.

FIG. 3 is a top perspective view of the outward-facing side of the open absorbent article.

FIG. 4 is a cross-sectional view of the extensible hip panel.

FIG. 5 is a side view of the absorbent article on a body.

FIG. 6 is a cross sectional view of the joining of the extensible hip panel to the main body of the absorbent article.

### DETAILED DESCRIPTION OF THE PREFERRED EMBODIMENTS

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, and below may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

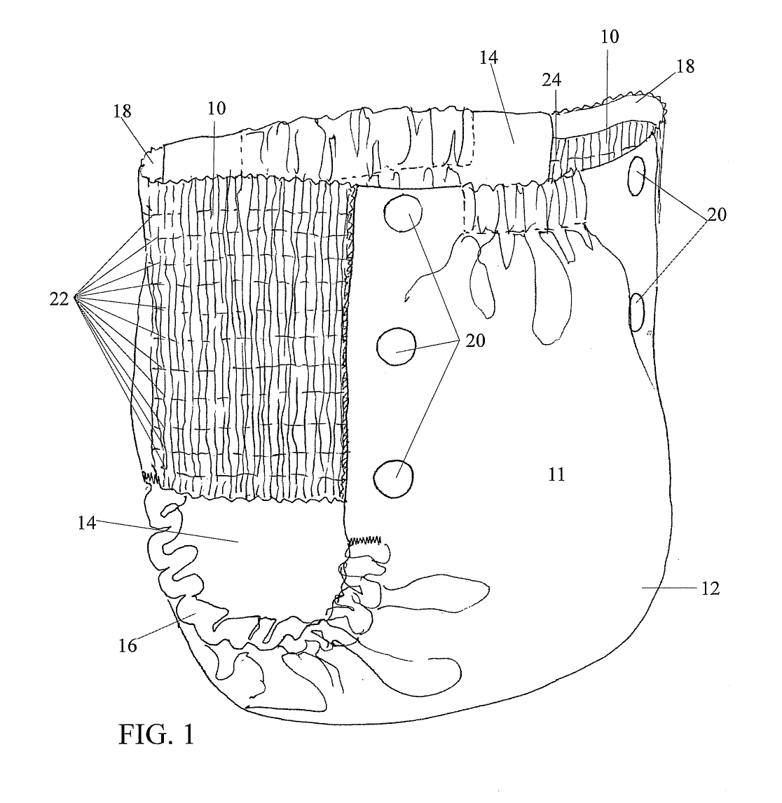

FIG. 1 is a perspective view of the absorbent article prepared for step-in donning. It shows a three-quarters view of the anterior and wearer's right lateral portion of the invention's preferred embodiment.

A pair of extensible hip panels 10 extend from the lateral posterior of a main diaper assemblage 11 and the opposing ends of said hip panels are removably affixed to the lateral anterior of the assemblage. The main diaper assemblage 11 comprises an impermeable exterior layer 12, a moisture transmitting skin-facing layer 14, and one or more layers of absorbent material 15 anchored between them.

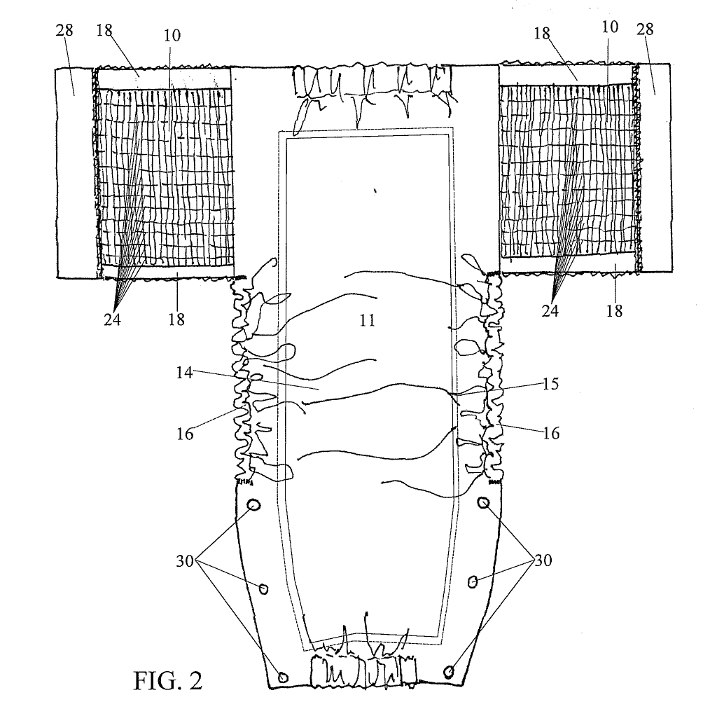

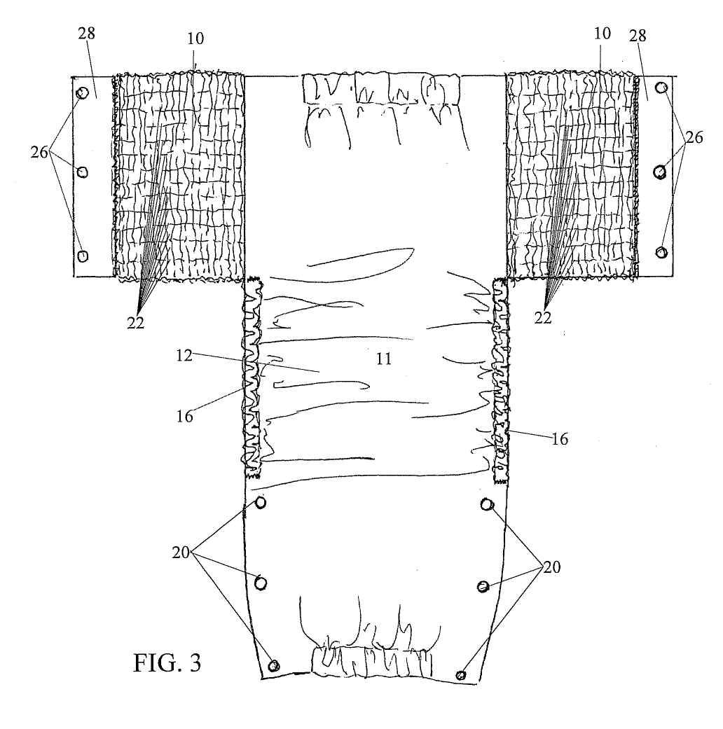

The impermeable exterior layer 12 is joined to the moisture transmitting skin-facing layer 14 at its entire perimeter, shown more clearly in FIG. 2 and FIG. 3, with a pair of groin-contouring elastic members 16 along the central portion of the main diaper assemblage 11 lateral edges.

FIG. 2 is a view from above of the skin-facing interior of the absorbent article. It shows a full view of the invention's preferred embodiment with the extensible hip panels 10 disconnected from the lateral anterior portion of the main diaper assemblage.

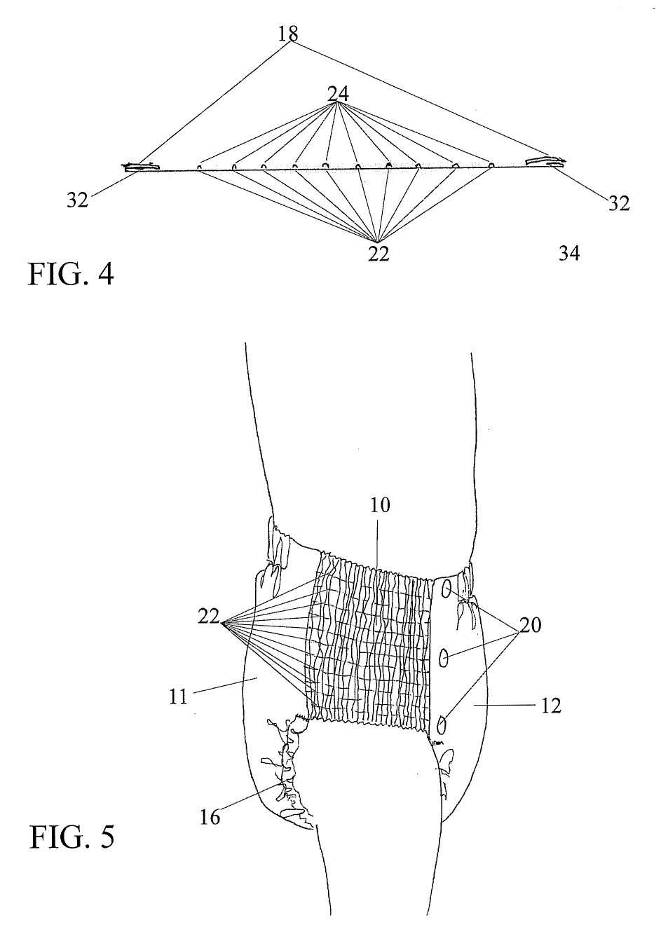

The extensible hip panels 10 are each constructed from a strip of woven material 34 and terminate in a snap facing 28. The strip of woven material 34 is rendered substantially elastic with a pair of edge stabilizing elastic members 18 joined over a pair of folded-under cut panel edges 32, visible in the cross-sectional view of FIG. 4, and a plurality of substantially parallel and evenly spaced strands of elastic thread 24 held in place with a plurality of substantially parallel and evenly spaced stitching 22, shown in FIG. 3.

FIG. 3 is a view from above of the exterior of the absorbent article. It shows a full view of the invention's preferred embodiment with the extensible hip panels 10 disconnected from the lateral anterior of the main diaper assemblage. A plurality of snap fastener prongs are affixed to the pair of snap facings 28 through only the outward-facing layer by preference, with a plurality of snap fastener caps 20, not visible, located inside the snap facings 28 piercing through to the exterior of the snap facings.

The plurality of substantially parallel and evenly spaced stitching 22 runs perpendicular to the main diaper assemblage 11 along the extensible hip panels 10 and the rows of elastic thread 24 on the interior face of the pair of hip panels 10, shown in FIG. 2, are securely caught at the posterior end in a seam with the lateral posterior of the main diaper assemblage and at the anterior end in a seam with a snap facing 28.

At FIG. 4 there is shown a cross-sectional view of the extensible hip panel 10. This drawing shows approximate positioning of the rows of stitching 22 and correspondingly the rows of elastic thread 24 relative to the pair of edge stabilizing elastic members 18 on the strip of woven material 32.

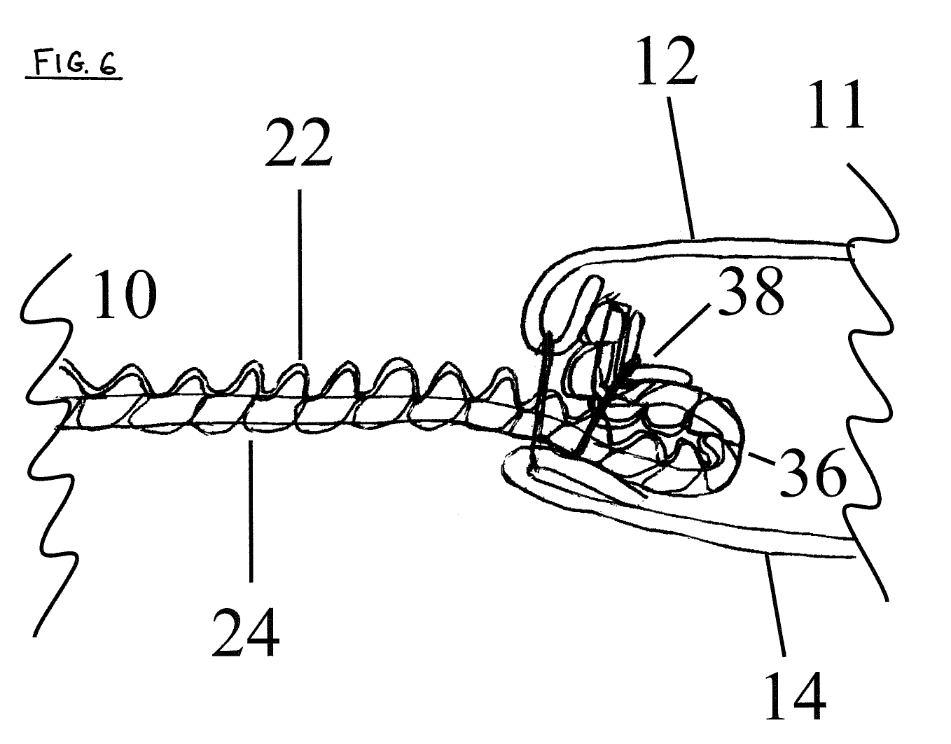

Turning to FIG. 5, there is shown a side view of the absorbent article as worn. For use as an absorbent article, a plurality of snap fastener prongs 26, as shown in FIG. 3, located on the pair of snap facings 28, connect the free ends of the extensible hip panels 10 to a plurality of snap fastener sockets 30 located on the main diaper assemblage 11 along the anterior portion of its lateral edges. A plurality of snap fastener caps 20 pass through the main diaper assemblage 11 and affix the plurality of snap fastener sockets in place.

The extensible hip panels 10 are capable of substantial deformation and recovery with minimal stress. A user of the absorbent article can choose to don the article by placing each foot into the corresponding leg opening while in an upright position, with the snap fastener prongs 26 and the snap fastener sockets 30 fastened and pull upward on the article until it is correctly positioned about the lower torso with the groin-contouring elastic members 16 in substantial contact with the user's skin along the groin region. A user can also choose to don the article in the manner of a conventional diaper, whether in an upright or prone position, with the snap fastener prongs 26 and the snap fastener sockets 30 disjoined while the article is passed through the user's legs, then joined together to secure the article in place.

The absorbent article is worn about the user's lower torso until it is time to go to the toilet or to change a wet or soiled diaper, at which time it is doffed either over the feet or by unfastening the snap fastener prongs 26 from the sockets 30 and passing the article from between the user's legs. Either manner of doffing is possible in either an upright or prone position.

After wearing, the absorbent article is laundered by conventional means with the skin-facing layer 14 reversed to be at the outside of the diaper.

The stretch elements such as the edge stabilizing elastic members 18 and rows of elastic thread 24 create a substantial amount of circumferential stretch in this absorbent article, allowing for easy pull-on donning and comfortable fit on users with a variety of high hip and abdominal girths, irrespective of the circumference of the user's low hip and upper thigh.

The groin-conforming elastic members 16 create a vertical elasticity toward the user's waist, pulling the central lateral edges of the main diaper assemblage 11 into substantial contact with the user's skin in order to contain most leaks effectively. The moisture impervious external layer 12 of the main diaper assemblage 11 also blocks moisture from escaping the enclosed absorbent layers 15 and leaching into the user's clothing.

Turning then to FIG. 6, there is shown a cross sectional view of the joining of the extensible hip panel 10 to the main diaper assemblage 11.

The extensible hip panel 10 may be comprised of a knit or non-woven material. Additionally, the extensible hip panels 10 may be rendered elastic with elastic cord instead of elastic thread. In the embodiment depicted in FIG. 6, knit or non-woven material is lined with rows of elastic threads 24 which are secured to the material using a plurality of substantially parallel and evenly spaced stitching 22. The stitching can be attached with any type of method, including, but not limited to, a lockstitch, a zigzag stitch, or, a chainstitch.

The edge of the extensible hip panel 10 is extended between the exterior layer 12 and the skin-facing layer 14 of the main diaper assemblage 11. The edge is then crimped 36 and held in place with reinforcing seam tape 38 which is stitched to the crimp so as to provide a non-slip seal.

Additional embodiments of the present invention can be configured in such a way that: the exterior layer 12 can be moisture permeable; the skin-facing layer 14 can be moisture diffusing instead of moisture transmitting; or, the absorbent layers 15 can be arranged in any other configuration. For example, the skin-facing layer 14 may be fabricated from an absorbent material; there may be a single hidden layer of absorbent material 15; or, there may be no hidden layer of absorbent material 15.

There may be an additional means of fastening laid-in absorbent materials 15, such as with a slit, or opening, in the skin-facing layer 14 for non-affixed absorbent layers to be inserted beneath it.

The extensible hip panels 10 may be constructed using material with elastic stretch rather than applied elastic thread; or, may be fabricated using a knit or non-woven material. Additionally, the extensible hip panels 10 may be rendered elastic with elastic cord instead of elastic thread. Or, the two ends of each of the extensible hip panels 10 may be permanently joined to both the anterior and posterior lateral edges of the main diaper assemblage 11. In another embodiment, there may be no edge-stabilizing elastic members 18 along the edges of extensible hip panels 10. The raw edges of the strip of woven material 34 may be finished in another manner. The facings 28 at the free ends of hip panels 10 may be fastened to the main diaper assemblage 11 with hook and loop tape or any other means.

The groin shaping may be achieved with other means than elastic casings, such as with the use of elastic trim, or with elastic stitched to the seam allowance.

The main diaper assemblage may include an additional dimensional leg cuff portion in proximity to each lateral edge.

The rows of elastic threads 24 may be attached with any type of method, including but not limited to lockstitch, couching in a zigzag stitch, inserting in a chainstitch, as the needle thread in a single-needle chainstitch, fusing, welding, melt bonding, or any other means.

The snap caps 20 may be visible or hidden between the layers of the main diaper assemblage 11 and the snap facings 28.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

What is claimed is:

1. A reusable, absorbent article for use by an ambulatory user, said article comprising:
(a) a main article assemblage having a posterior end and an anterior end opposed to said posterior end;
(b) a first extensible hip panel attached to the right side of said posterior end; and
(c) a second extensible hip panel attached to the left side of said posterior end.

2. The article of claim 1, wherein said main article assemblage further comprises:
(a) an impermeable exterior layer;
(b) a moisture transmitting, skin facing layer; and
(c) one or more layers of absorbent material secured between said impermeable exterior layer and said moisture transmitting skin facing layer.

3. The article of claim 2, wherein said impermeable exterior layer is joined to said moisture transmitting skin facing layer along the entire perimeter of said moisture transmitting skin facing layer.

4. The article of claim 3, wherein a portion of said entire perimeter is joined with a first groin contouring elastic member along a first lateral edge and a second groin contouring elastic member along a second lateral edge of the central portion of said main article assemblage.

5. The article of claim 1, wherein said first and said second hip panels each further comprise:
(a) a strip of woven material;
(b) a cut panel edge;
(c) a plurality of substantially parallel and evenly spaced strands of elastic thread, secured in place by a plurality of substantially parallel and evenly spaced stitching, wherein each of said stitches corresponds to each of said threads; and
(d) an edge stabilizing elastic member.

6. The article of claim 5, wherein said plurality of substantially parallel and evenly spaced stitching runs perpendicular to said main article assemblage.

7. The article of claim 1, wherein said first and said second hip panels each further comprise a first and a second corresponding snap facing attached to the edge opposed to the main article assemblage of each of said first and said second hip panel.

8. The article of claim 7, wherein said substantially parallel and evenly spaced strands of elastic thread on the interior face of each of said hip panels are secured at the posterior end in a seam with the lateral posterior of said main article assemblage; and, at the anterior end in a seam with said corresponding snap facing.

9. The article of claim 8, wherein one or more snap fastener prongs are affixed to each of said first and said second snap facings; and, one or more snap fastener sockets corresponding to said one or more snap fastener prongs, are located on said main diaper assemblage along the anterior portion of its lateral edges.

10. The article of claim 9, wherein each of said snap fastener prongs is affixed to a snap facing through only said outward facing layer.

11. A reusable, absorbent diaper for easy donning and doffing by a user, said diaper comprising:
(a) a main article assemblage having a posterior end and an anterior end opposed to said posterior end;
(b) a first extensible hip panel attached to the right side of a selected said posterior end or a selected said anterior end;
(c) a second extensible hip panel attached to the left side of said selected posterior end or said selected anterior end; and
(d) one or more snap fastener caps located toward the outer lateral edge of said posterior end or said anterior end opposite said selected posterior end or said selected anterior end of said main article assemblage.

12. The diaper of claim 11, wherein said main article assemblage further comprises:
(a) an exterior layer;
(b) a skin facing layer; and
(c) one or more layers of absorbent material secured between said exterior layer and said skin facing layer.

13. The diaper of claim 12, wherein said exterior layer is joined to said skin facing layer along the entire perimeter of said skin facing layer.

14. The diaper of claim 13, wherein a portion of said entire perimeter is joined with a first groin contouring elastic member along a first lateral edge and a second groin contouring elastic member along a second lateral edge of the central portion of said main article assemblage.

15. The article of claim 11, wherein said first and said second hip panels each further comprise:
(a) a strip of woven material;
(b) a cut panel edge;
(c) a plurality of substantially parallel and evenly spaced strands of elastic thread, secured in place by a plurality of substantially parallel and evenly spaced stitching, wherein each of said stitches corresponds to each of said threads; and
(d) an edge stabilizing elastic member.

16. The article of claim 15, wherein said plurality of substantially parallel and evenly spaced stitching runs perpendicular to said main article assemblage.

17. The article of claim 11, wherein said first and said second hip panels each further comprise a first and a second corresponding snap facing attached to the edge opposed to the main article assemblage of each of said first and said second hip panel.

18. The article of claim 17, wherein said substantially parallel and evenly spaced strands of elastic thread on the interior face of each of said hip panels are secured at the posterior end in a seam with the lateral posterior of said main article assemblage; and, at the anterior end in a seam with said corresponding snap facing.

19. The article of claim 17, wherein one or more snap fastener prongs are affixed to each of said first and said second snap facings.

20. The article of claim 17, wherein one or more snap fastening sockets, corresponding to said one or more snap fastener prongs, are located on said main diaper assemblage along the anterior portion of its lateral edges.

21. A reusable, absorbent article for use by an ambulatory user, said article comprising:
(a) a main article assemblage, said main article assemblage further comprising:
(i) a posterior end and an anterior end opposed to said posterior end; and
(ii) an outermost layer and a skin-facing layer;
(b) a first extensible hip panel attached to the right side of said posterior end;
(c) a second extensible hip panel attached to the left side of said posterior end; and
(d) wherein each of said first extensible hip panel and said second extensible hip panel are attached to said main diaper assemblage by corresponding attachment means, said corresponding attachment means further comprising:
(i) a crimped edge of said first or said second extensible hip panel passing between said outermost layer and said skin-facing layer of said main diaper assemblage; and
(ii) reinforcing seam tape stitched to said crimped edge.

22. The reusable, absorbent article of claim 21, wherein:
(a) said outermost layer is an impermeable layer;
(b) said skin-facing layer is a moisture transmitting layer; and
(c) one or more layers of absorbent material are secured between said impermeable outermost layer and said moisture transmitting skin-facing layer.

23. The reusable, absorbent article of claim 22, wherein said impermeable outermost layer is joined to said moisture transmitting skin-facing layer along the entire perimeter of said moisture transmitting skin facing layer.

24. The reusable, absorbent article of claim 23, wherein a portion of said entire perimeter is joined with a first groin contouring elastic member along a first lateral edge and a second groin contouring elastic member along a second lateral edge of the central portion of said main article assemblage.

25. The reusable, absorbent article of claim 21, wherein said first and said second hip panels each further comprise:
(a) a strip of woven material;
(b) a cut panel edge;
(c) a plurality of substantially parallel and evenly spaced strands of elastic thread, secured in place by a plurality of substantially parallel and evenly spaced stitching, wherein each of said stitches corresponds to each of said threads; and
(d) an edge stabilizing elastic member.

26. The reusable, absorbent article of claim 25, wherein said plurality of substantially parallel and evenly spaced stitching runs perpendicular to said main article assemblage.

27. The reusable, absorbent article of claim 21, wherein said first and said second hip panels each further comprise a first and a second corresponding snap facing attached to the edge opposed to the main article assemblage of each of said first and said second hip panel.

28. The reusable, absorbent article of claim 27, wherein said substantially parallel and evenly spaced strands of elastic thread on the interior face of each of said hip panels are secured at the posterior end in a seam with the lateral posterior of said main article assemblage; and, at the anterior end in a seam with said corresponding snap facing.

29. The reusable, absorbent article of claim 28, wherein one or more snap fastener prongs are affixed to each of said first and said second snap facings; and, one or more snap fastener sockets corresponding to said one or more snap fastener prongs, are located on said main diaper assemblage along the anterior portion of its lateral edges.

30. A reusable, absorbent article for use by an ambulatory user, said article comprising:
(a) a main article assemblage having an anterior end and a posterior end;
(b) a first extensible hip panel attached to a first side of said posterior end;
(c) a second extensible hip panel attached to a second side of said posterior end; and
(d) first attachment means for attaching said first extensible hip panel to said first side of said posterior end; and
(e) second attachment means for attaching said second extensible hip panel to said second side of said posterior end.

31. The reusable, absorbent article of claim 30, wherein said main article assemblage further comprises:
(a) an outermost layer; and
(b) a skin-facing layer.

32. The reusable, absorbent article of claim 31, wherein each of said first and said second attachment means comprises:
(a) a crimped edge of said first or said second extensible hip panel passing between said outermost layer and said skin-facing layer of said main diaper assemblage; and
(b) reinforcing seam tape stitched to said crimped edge.

33. The reusable, absorbent article of claim 31, wherein:
(a) said outermost layer is an impermeable layer;
(b) said skin-facing layer is a moisture transmitting layer; and
(c) one or more layers of absorbent material are secured between said impermeable outermost layer and said moisture transmitting skin-facing layer.

34. The reusable, absorbent article of claim 33, wherein said impermeable outermost layer is joined to said moisture transmitting skin-facing layer along the entire perimeter of said moisture transmitting skin facing layer.

35. The article of claim 23, wherein a portion of said entire perimeter is joined with a first groin contouring elastic member along a first lateral edge and a second groin contouring elastic member along a second lateral edge of the central portion of said main article assemblage.

36. The article of claim 31, wherein said first and said second hip panels each further comprise:
(a) a strip of woven material;
(b) a cut panel edge;
(c) a plurality of substantially parallel and evenly spaced strands of elastic thread, secured in place by a plurality of substantially parallel and evenly spaced stitching, wherein each of said stitches corresponds to each of said threads; and
(d) an edge stabilizing elastic member.
